(19) United States
(12) Patent Application Publication
(10) Pub. No.: US 2019/0336350 A1
(43) Pub. Date: Nov. 7, 2019

(54) INTEGRAL PROTECTOR FOR A LIVING BEING MEMBER

(71) Applicants: FUNDACIÓ EURECAT, Cerdanyola del Valles BA (ES); FUNDACIÓ SALUT DEL CONSORCI SANITARI DEL MARESME, Mataro BA (ES)

(72) Inventors: Jesús ALÓS VILLACROSA, Sant Vicenç de Montalt BA (ES); Francesc PERA ISERN, Calella BA (ES); Xavier VILÀ ALFONSO, Canet de Mar BA (ES); Jordi PLANAGUMÀ VILÀ, Mataró BA (ES); Carles PÉREZ SAEZ, Mataró BA (ES); Daniel ROIG BARDINA, Canet de Mar BA (ES); Jaume ROSELL PEREA, Canet de Mar BA (ES); Miquel SOLER LUQUE, Canet de Mar BA (ES); Virginia GARCIA CANDEL, Sta. Perpètua de la Mogoda BA (ES)

(21) Appl. No.: 16/305,176

(22) PCT Filed: May 30, 2017
(86) PCT No.: PCT/IB2017/053158

§ 371 Date: Nov. 28, 2018

(30) Foreign Application Priority Data
May 30, 2016 (EP) 16382240.6

(51) Int. Cl. A61F 13/06; D04B 1/22; D04B 1/26

(52) U.S. Cl. CPC A61F 13/069; D10B 2501/043; D04B 1/265; D04B 1/225

(57) ABSTRACT

The integral protector comprises a tubular assembly (16) obtained from a textile tubular stockinette (14), covering the member and an external tubular jacket (10) coaxially joined and providing protection and padding effects. The textile stockinette (14) has an elastic longitudinal portion (15) extending along a generatrix thereof and the tubular jacket (10), receiving placed therein said textile stockinette (14) is made of a 3D spacer fabric

### FIELD OF THE INVENTION

The invention pertains to field of the devices for protecting by cushioning the skin of a patient in particular against foot and heel pressure ulcers that are a major healthcare problem.

Prevalence of foot ulcers among elderly people admitted to general hospitals or nursing homes is 28-35% and could be prevented by this protector. This will improve the health status and the quality of living of these frail patients and reduce healthcare expenses.

Foot and heel pressure ulcers are a major healthcare problem. A pressure ulcer is an injury to the skin, usually over a bony prominence, as a result of constant pressure due to impaired mobility (because of wheelchair-bound or bed-bound). The pressure results in reduced blood flow and eventually may cause cell death, skin breakdown, and the development of an open wound. Common sites include the sacrum (tailbone), lower back, back of the head, elbow or heels. The heel is the second most common anatomical location for pressure ulcers after sacrum. Prevalence of heel pressure ulcers has been estimated between 18 and 28% among adult population in acute care, long-term care and homecare settings. Healing times for pressure ulcers are long, in many cases over a year. If the conditions leading to the pressure sore are not rapidly corrected, the localized skin damage will spread to deeper tissue layers affecting muscle, tendon, and bone. If not adequately treated, open ulcers can become a source of pain, disability, and infection. Moreover, amputation due to persisting infection or non-healing heel ulcers may occur in up to 40% of cases.

The cause of the pressure ulcer is the reduction of the blood flow in the point of pressure which carries death cell and ulceration. The heel is at increased risk of ulceration as a result of its posterior prominence and lack of padding over the calcaneus. The hyperaemic response to pressure loading on the heel does not differ from other tissue. However, with ageing the number of capillaries is reduced, the amount of soft-tissue padding over the calcaneus decreases and blood flow at rest to the heel is relatively low. Blood flow to the heel comes through the peroneal and posterior tibial arteries, which are often affected by peripheral arterial disease. Due to the unique anatomic characteristics of the heel and its impaired ability to reperfuse, the heel is a common site for deep tissue injury. Apart from immobilization and bed confinement, main risk factors for pressure ulcers include:
a) diseases impairing sensation such as diabetic neuropathy, stroke, and nerve block after surgery or analgesia,
b) conditions that reduce blood flow to the leg such as peripheral vascular disease (with ankle-brachial indices lower than 0.8) or vasopresive medications,
c) respiratory diseases that reduce tissue oxygenation,
d) lower limb weakness due to hip fracture or total knee replacement,
e) malnutrition and low serum albumin levels,
f) diabetes, and
g) ageing.

In addition, secondary (sometimes iatrogenic) foot ulcers can occur as a consequence of mishandling of heel ulcers

### BACKGROUND OF THE INVENTION

Prevention of pressure ulcers is a key issue because treatment can be difficult and established ulcers can result in devastating consequences such as amputation or even death. Prevention of pressure ulcers is largely based upon the principle of offloading and ensuring no skin areas exposed to pressure and shear forces. All bedridden patients need to be considered at risk of pressure ulcers and must follow universal precautions including: daily inspection of feet and heels and skin integrity, assessment of quality of pulses in the foot and ankle, maintain the skin clean and moisturized by applying cream/lotion every day, float heel from the bed, prevent friction injury by applying film/foam dressings, frequent careful changing of body position (turning bedridden patients every two hours to remove pressure), get patient out of bed as soon as possible or two times a day is possible, use of special mattresses or local protection supports, use of protectors, motion exercises, management of other contributing illnesses, or implementation of a healthy diet. Relieving or reducing the pressure on the area is essential. Offloading devices should completely lift the heel from the bed. The use of pillows or boots is common. Foam mattresses are able to reduce pressure ulcer incidence. Pillows can be used in patients who are not moving their legs, but boots are needed for patients who move about in bed or who are at high risk for ulceration. Several offloading boots have showed effectiveness in reducing pressure ulcers incidence but none of them have demonstrated to be superior respect the others.

It is considered essential to decrease the pressure in the main risk areas of the foot to prevent pressure ulcers. Thus, to “float” the foot by placing a pillow under the calves or using boots or other devices that lift the heel from the bed is considered an indispensable preventing measure. However, too often the pillow move and is not located where it is needed to be. Although protecting foot devices move less than pillows, sometimes protecting devices are not well fastened and also move. It is not rare to observe these devices in a wrong position or place so they cease to have any effect. It's very important to guarantee that the device remain in place while the patient is moving the leg to avoid shear forces. On the other hand, some devices offer only partial protection, protecting only the heel and/or the ankle, but leaving other areas unprotected and exposed to pressure, friction and shear. Even more, some of these devices may incur insignificant iatrogenic ulcers due to the pressure exerted on the back of the foot or may incur in other complications such as deep vein thrombosis or Achilles tendon injury.

Many devices for the treatment of ulcers are known.

GB 2465350 discloses a generally tubular sock having a retaining means such as a pocket for connecting a support member to the sock, said support member being of a size sufficient to extend between the rear portion of the lower leg and a surface on which the subject is lying such that the heel of the subject is held above and out of contact with the surface.

DE 202005009506U1 refers to a foot bandage for preventing pressure sores in patients sleeping on side or back and includes a continuous elastic woven fabric with incorporated foam or gel cushion.

WO 2009015418 discloses a skin protector for a joint region comprising a fabric sleeve having and end portion and an opposite end portion joined by an intermediate portion which contains a bend whereby a waterproof cushioning pad or disc may be provided containing silicone gel or other suitable materials attached to said sleeve.

On the other hand U.S. Pat. No. 7,465,283 describes a cast or support assembly comprising a compressible padding layer including a padding material formed of double knit type fabric material having an inner layer of finely woven or knit material with openings defining a smooth surface structure on the side to face the skin, and an outer layer of coarsely woven or knit material with openings therethrough spaced from said inner layer by an open work matrix of filaments which are integrally woven or knit into said inner and said outer layers of woven or knit material, the outer layer openings being larger than the openings of the inner layer, the padding layer being devoid of a hardening material such that the padding layer remains compressible upon exposure to water. Additional casting fabric is also provided, with this casting fabric being impregnated with water hardenable material.

A similar solution to the last referred patent is used in U.S. Pat. No. 6,981,955 relating to a castliner for a patient and particularly to a sleeve-like knitted structure, for use as a castliner having superior cushioning and enhanced comfortable conformance to the body.

U.S. Pat. No. 8,790,297 discloses an orthopaedic padding including a knitted elongate spacer fabric defining a first and second opposing faces each having pillar inlay stich construction interconnected and separated from one another through an intermediate spacer area having a needle V stich construction, wherein the orthopaedic padding is an undercast liner.

Also EP 1082075 relates to a hardenable orthopaedic support comprising a double layer fabric in the form of a flat sheet which was impregnated with a water-hardenable material and having spaced interwoven layers and an open work matrix of filaments of fibres interconnecting said layers.

The present invention aims a new innovative concept of an integral protector for a patient member, in particular for a foot, providing a total protection of all the potential foot areas at risk against pressure ulcers (heel, back, sides) based on the principle of offloading or cushioning and ensuring no skin areas exposed to pressure and shear forces.

### DISCLOSURE OF THE INVENTION

In order to solve the above mentioned objective, i.e. to improve the prevention of heel pressure ulcers and overcome the limitations of current solutions, the invention proposes developing a one-piece boot/stocking for integral foot protection in which a 3D fabrics as padding or cushioning material is used surrounding an inner textile tubular envelope of the member intended to be in contact with the skin, the assembly being able to successfully overcome functional limitations associated with current models of heel protectors on the market.

3D spacer fabric technology fits with the main drawbacks in that has to be overcome with the integral protector proposed in terms of relieving or reducing the pressure on the foot area. Textile technology will also allow the design of a product easy to use, comfortable and well fastened avoiding the exposure to friction and shear.

As known 3D fabric is a textile structure made of 2 fabrics (A and B) joined together through a monofilament which supports the 3D structure and provides higher or lower rigidity. Further, the distance between A and B fabrics can be adjusted depending on the final application. Fabrics can be subject to specific finishes in order to enhance its performance in contact with skin and the comfort of the user.

The integral foot protector of the invention wants to be a new health device to be worn in the foot to prevent pressure ulcers in high risk patients.

According to the invention the integral protector against foot and heel pressure ulcers of a patient comprises a one-piece tubular assembly consisting of:
a textile tubular stockinette acting as a tubular envelope in contact with the skin of the member, said textile stockinette being either knitted, woven or braided, said textile stockinette comprising at least an elastic longitudinal portion, provided when knitting said stockinette and extending along a whole generatrix thereof, and
a padding in the form of a tubular jacket made of a 3D spacer fabric providing an external sleeve, said tubular jacket receiving placed therein said textile stockinette arranged coaxial and covering an inner surface thereof, the textile stockinette being attached to said tubular jacket so that it cannot move relative thereto.

Thereby the tubular jacket operates by decreasing the pressure acting on the cited living being member when in rest against a surface.

In an embodiment the referred elastic longitudinal portion of the textile tubular stockinette is a strip of regular width.

According to a preferred embodiment the inner textile tubular stockinette and the tubular envelope are joined at least by means of a seam of the mouthpiece area of both elements the cited seam of the mouthpiece being preferably done on an external peripheral zone of the tubular jacket in order not to affect the skin of a patient.

The tubular jacket can be obtained from a flat piece with two opposite cut-out portions by joining two opposite major sides thereof, said cut-out portions providing an angled or bent portion corresponding to an elbow of the tubular jacket suitable to accommodate against the external surface of a foot.

The cited angled or bent portion is obtained by means of seam of the edges of said cut-out portion in a middle region of the tubular jacket forming an angle with the axis thereof

In an embodiment the textile stockinette is further attached to said tubular jacket by means of said elastic longitudinal portion being stitched to the facing major edges of the cited flat piece forming the tubular jacket

To facilitate insertion of the foot the tubular assembly has an end portion wider than the opposite end. Further one of the ends of the tubular assembly includes a mark providing an indication of the introduction area of the member to protect.

In order to provide a reusable assembly both the textile stockinette fabric and the 3D spacer fabric are washable and breathable.

According to an additional embodiment the textile tubular stockinette is longer than the tubular jacket, and a protruding segment of said textile tubular stockinette protrudes from one mouthpiece area of said tubular jacket, and wherein the mouthpiece area of said textile tubular stockinette protruding segment is collapsed and sealed. In other words the textile tubular stockinette is a sock and the tubular jacket covers all but the protruding segment of said textile tubular stockinette which corresponds with the segment of the sock which covers the toes. This feature prevents the one-piece tubular assembly to be accidentally moved along the living being member away from the toes.

Preferably the collapsed mouthpiece area of said textile tubular stockinette protruding segment is sealed by sewing.

The present invention also proposes a topical drug being carried by the textile tubular stockinette producing a gradual release of said topical drug to the skin of the living being in contact with said the textile tubular stockinette. For example the topical drug can be a skin hydration agent.

According to a preferred embodiment said topical drug is a cream, lotion, gel, foam, or ointment impregnated to the textile tubular stockinette.

In addition is proposed the topical drug being encapsulated in micro or nano capsules supported on the textile tubular stockinette, said capsules releasing the topical drug under heat, rub or pressure.

### BRIEF DESCRIPTION OF THE DRAWINGS

FIG. 1 is a perspective view of the flat 3D spacer fabric intended to provide an external sleeve or tubular jacket by joining the two major edges thereof, and with two opposite cut-out portions that then provide a bent portion.

FIG. 2 is a perspective view of the textile tubular stockinette including an elastic longitudinal portion along the whole generatrix thereof.

FIG. 3 is a perspective view of the 3D spacer fabric with the cut-out portions sewn forming an angle o with the axis thereof, providing an elbow portion.

FIG. 4 is a perspective view of the textile tubular stockinette positioned for assembly.

FIG. 5 is a perspective view of the integral protector of the invention with the textile stockinette attached to the external 3D spacer fabric sleeve.

FIGS. 6 to 10 illustrate several spacer textiles known in the art that have been tested in order to find a best 3D fabric for the production of the tubular jacket or the integral protector of this invention, using fabrics with following weights and thicknesses:

FIG. 6: Spacer fabric 1 Weight 330 g/m² Thickness 3.5 mm

FIG. 7: Spacer fabric 2 Weight 220 g/m² Thickness 6 mm

FIG. 8: Spacer fabric 3 Weight 230 g/m² Thickness 4.2 mm

FIG. 9: Spacer fabric 4 Weight 270 g/m² Thickness 3.2 mm

FIG. 10: Spacer fabric 5 Weight 310 g/m² Thickness 3.5 mm

FIG. 11 is a photography of the one-piece tubular assembly according to an alternative embodiment in which the textile tubular stockinette is longer than the tubular jacket, being a protruding segment of said textile tubular stockinette collapsed and sealed.

### DETAILED DESCRIPTION OF THE INVENTION

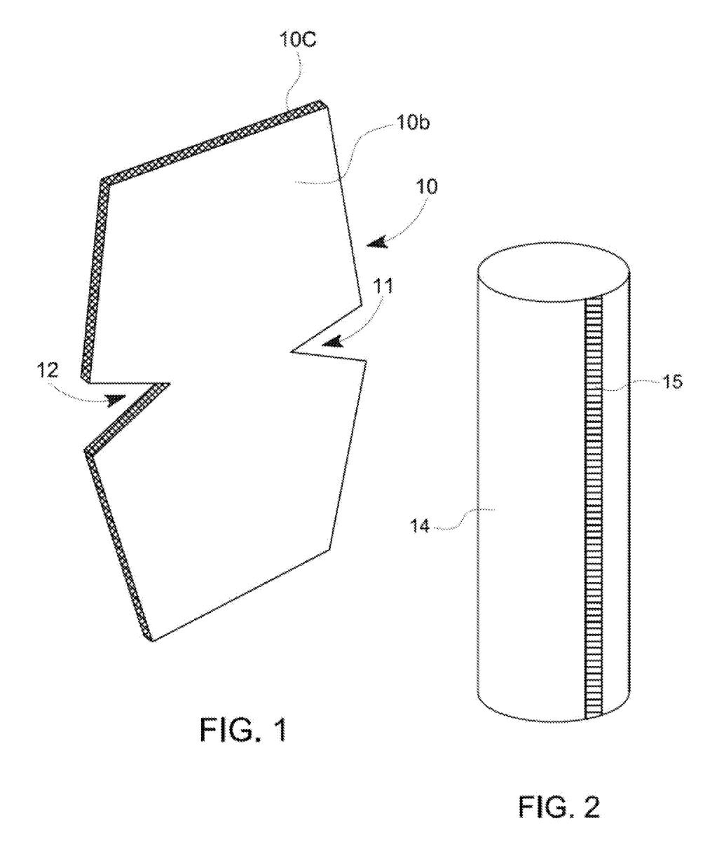

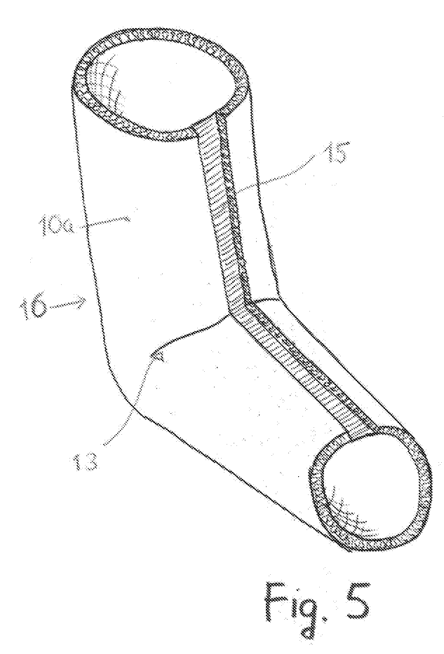

FIG. 1 details a flat piece of a 3D-spacer fabric 10 including a double layer fabric 10a, 10b with an open-work of filaments or fibres 10c interconnecting said layers 10a, 10b, The flat piece of 3D-spacer fabric 10 includes along two opposite major edges two opposite cut out portions 11, 12, that when these major edges and the edges of the cut out portions 11, 12 are joined (see FIG. 5) by stitching o sewing provide a bent part 13 conforming an elbow.

FIG. 2 illustrates a textile tubular stockinette 14 which makes the interior part of the tubular assembly constituting the proposed integral protector. In the figure it has been indicated an elastic longitudinal portion 15, extending along a whole generatrix of the stockinette 14, provided while knitting the stockinette, in the form of a strip of regular width. This elastic strip provides a means allowing that the stockinette acts as a tubular envelope tightly overlapped on the section of the member to be protected, for example a joint region of a foot.

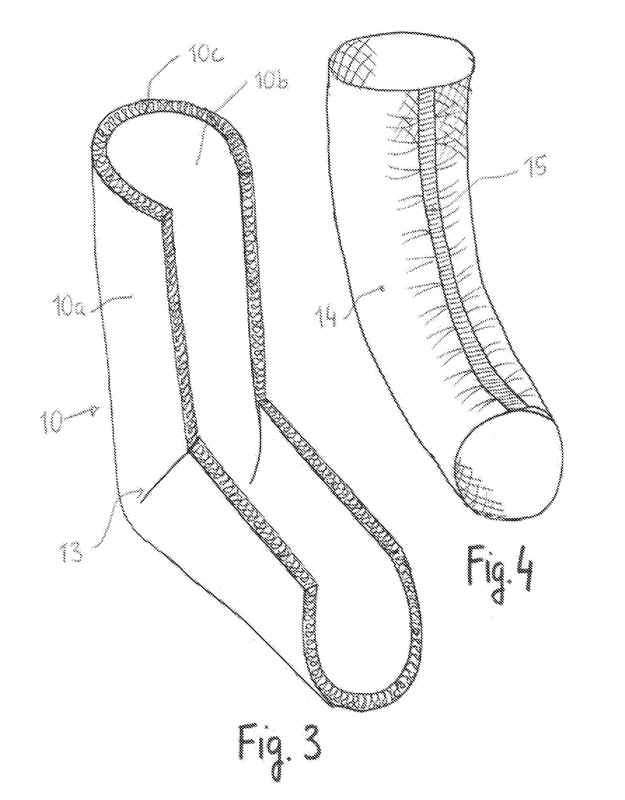

FIGS. 3 and 4 show the 3.D-spacer fabric 10, preconfigured in a non closed tubular shape prepared to receive inside the stockinette 14, and with the edges of the cut out portions 11, 12, already joined by stitching providing a bent of and elbowed portion. The stockinette 14 is arranged inside the tubular jacket or external sleeve 10, so that extending coaxially within thereof, and joined by sewing of the major edges of the external sleeve 10 along the elastic longitudinal portion 15, providing in this way an elastic adaptation of the one-piece tubular assembly 16 (see FIG. 5) surrounding the part of the member to be covered procuring a cushioning effect (3-D-spacer fabric 10) and a protection of the skin of the patient by the inner textile tubular stockinette 14.

In addition to the joining by means of the cited sewing of the major edges of the external sleeve 10 along the elastic longitudinal portion 15, a further union between both stockinette 14 and tubular jacket 10 can be provided by providing a seam of the mouth piece area of both elements 10, 14 when shaped as a tubes. The joining is performed preferably on an external peripheral zone of the tubular jacket 10 to avoid any incidence on the skin of the patient. Thereby it is ensured that the textile tubular stockinette 14 will remain attached to the inner wall of the tubular jacket 10, without forming wrinkled areas, and then providing a suitable protection of the patient skin.

Moreover both said 3-D spacer fabric of the tubular jacket 10 and the knitted fabric of the stockinette 14 are washable and breathable so that providing a reusable integral protector.

At least when the proposed integral protector is intended for a foot and heel pressure ulcers protection, the one-piece tubular assembly of the two joined elements 10 and 14 will have an end portion wider that the other end and a suitable mark will be included in the protector to indicate the area of insertion of the member to protect.

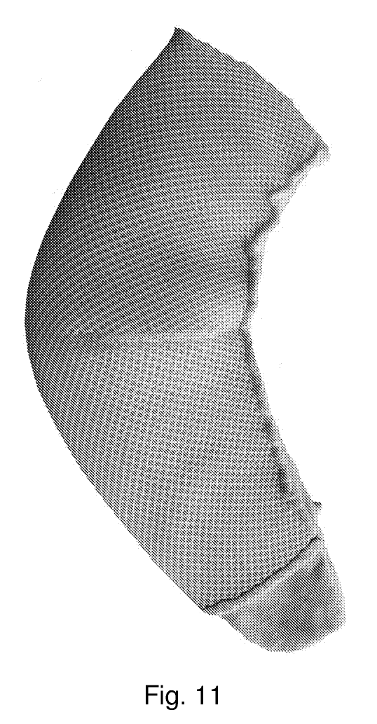

FIG. 11 shown an alternative embodiment in which the textile tubular stockinette is longer than the tubular jacket, being the a tubular jacket end, corresponding with one of its mouthpiece area, collapsed and sealed by sewing.

### Experimental Tests

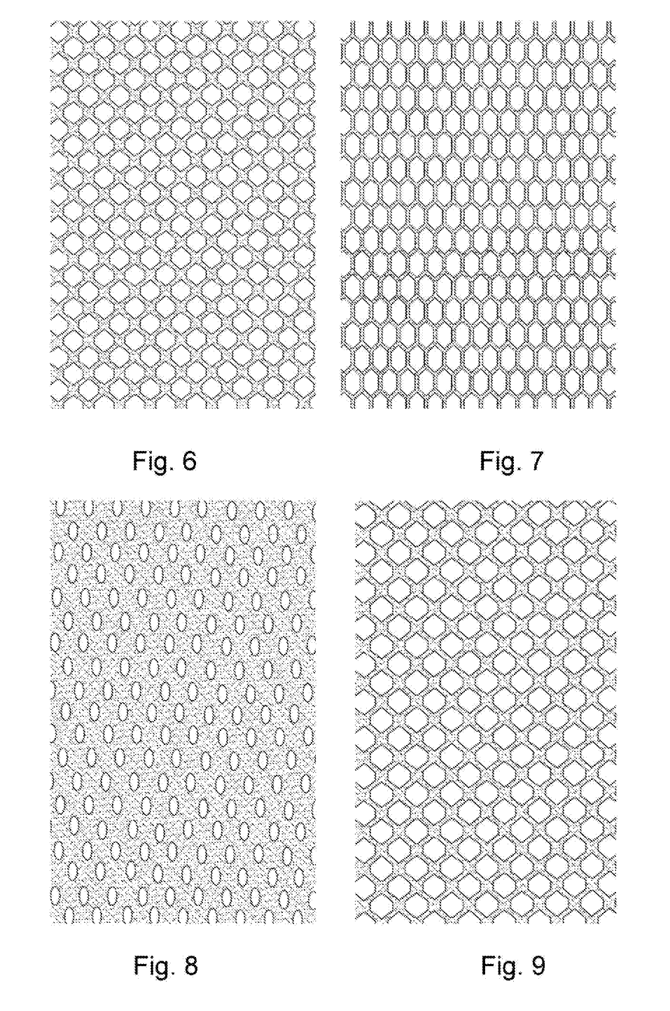

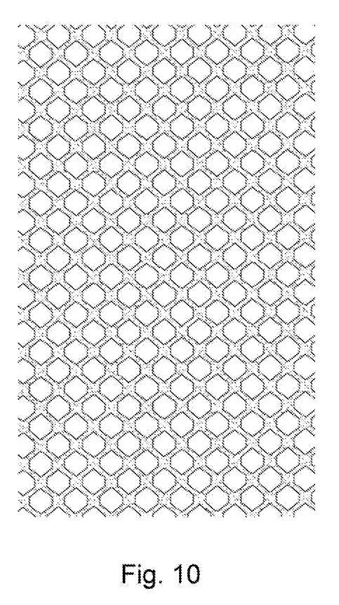

Five 3D spacer textiles known in the art (see corresponding pictures and technical details on FIGS. 6 to 10) have been tested in order to finding a best 3D fabric for the production of the integral protector.

Two methods have been used to perform the test, a direct method using real patients and a dynamometric method using a series of weights (simulating a foot) programmed on a dynamometer.

The process consisted in marking the foot (real or artificial) with an iodine solution and then letting it rest during 4 seconds over a friseline sheet resting over the 3D fabric.

This process is repeated 29 times on each one of the 3D spacer textiles initially tested (also without a textile) and in random order. A foot mark appears visible on the friseline sheet depending on the cushioning of the texted 3D spacer.

1. Integral protector for a living being member including a foot for prevention of pressure ulcers, comprising a one-piece tubular assembly (16) consisting of:
a textile tubular stockinette (14) providing a tubular envelope intended to be in contact with the skin of the living being, said textile stockinette (14) comprising at least an elastic longitudinal portion (15) extending along a whole generatrix thereof, and
a tubular jacket (10) made of a 3D spacer fabric providing an external sleeve, said tubular jacket receiving placed therein said textile stockinette (14), arranged coaxially and covering an inner surface thereof, said textile stockinette (14) being attached to said tubular jacket,
wherein said tubular jacket (10) operating as padding by decreasing the pressure acting on the cited living being member when in rest against a surface, said stockinette (14) acting as a skin protector and the one-piece tubular assembly providing a total protection of all the potential living member areas at risk against pressure ulcers.

2. Integral protector, according to claim 1, wherein said attachment between said stockinette (14) and said tubular jacket (10) is obtained at least by means of a seam of the mouthpiece area of both elements so that the integral protector remaining in place while the living member is moving.

3. Integral protector according to claim 2, wherein the cited seam of the mouthpiece is done on an external peripheral zone of the tubular jacket (10).

4. Integral protector according to claim 1, wherein said textile stockinette is either knitted, woven or braided.

5. Integral protector, according to claim 1 wherein said tubular jacket (10) is obtained from a flat piece with two opposite cut-out portions (11, 12) by joining two opposite major edges of the flat piece, said cut-out portions (11, 12) when joined providing an angled portion corresponding to an elbow of the tubular jacket (10) suitable to accommodate against the external surface of a foot.

6. Integral protector, according to claim 1 wherein said tubular jacket (10) is obtained from a flat piece with two opposite cut-out portions (11, 12) by joining to said elastic longitudinal portion (15) of the stockinette (14), two opposite major edges of the flat piece, said cut-out portions (11, 12) providing an angled portion corresponding to an elbow of the tubular jacket (10) suitable to accommodate against the external surface of a foot.

7. Integral protector, according to claim 5, wherein said angled portion is obtained by means of a seam of the edges of said cut-out portions (11, 12) around a middle region of the tubular jacket (10) forming an angle with the axis thereof.

8. Integral protector, according to claim 1, wherein said elastic longitudinal portion (15) is provided while knitting said stockinette.

9. Integral protector, according to claim 1, wherein said elastic longitudinal portion (15) is a strip of regular width.

10. Integral protector, according to claim 1, wherein said tubular assembly (16) has an end portion wider than the opposite end.

11. Integral protector, according to claim 9, wherein one of the ends of the tubular assembly (16) includes a mark providing an indication of an introduction area of the member to protect.

12. Integral protector, according to claim 1, wherein the fabric of the textile stockinette (14) is washable and breathable.

13. Integral protector, according to claim 1, wherein the 3D spacer fabric of said tubular jacket (10) is washable and breathable.

14-19. (canceled)
